(19) United States
(12) Patent Application Publication
(10) Pub. No.: US 2019/0175106 A1
(43) Pub. Date: Jun. 13, 2019

(54) HEALTH AND ATHLETIC MONITORING SYSTEM, APPARATUS AND METHOD

(71) Applicant: Sivan Langer, Gesher Haziv (IL)

(72) Inventor: Sivan Langer, Gesher Haziv (IL)

(73) Assignee: SIVAN LANGER, Gesher Haziv (IL)

(21) Appl. No.: 16/223,222

(22) Filed: Dec. 18, 2018

Related U.S. Application Data
(60) Provisional application No. 62/557,798, filed on Sep. 13, 2017.

(51) Int. Cl. A61B 5/00; A61B 5/103; A61B 5/0205; A43B 17/00; A43B 3/00

(52) U.S. Cl. CPC A61B 5/6807; A61B 5/1038; A61B 5/746; A61B 5/747; A61B 5/04011; A61B 5/0022; A43B 17/00; A43B 3/0005; A61B 5/0205

(57) ABSTRACT

A health and athletic monitoring system apparatus. The apparatus is located in proximity to the feet and has an attached receiver/transmitter and a sensor to obtain signals from the individual's body. The sensor remote-senses the data via a material.

A method for tracking health via feet with aid of a given tracking device including measuring electrocardiographic signals via feet and remote sensing measurement. A system of maintain analyzing and predicting health and athletics related data and sharing it with one individual or a group of ones thereof.

### CROSS-REFERENCE TO RELATED APPLICATIONS

A previous application related to this by the assignee is U.S. provisional Application No. 62/557,798, filed Sep. 19, 2017, entitled ““Smart” Insole with a Heart Rate and speed/acceleration/distance/impact/landing angle sensors and transmitter Apparatus”.

### BACKGROUND OF THE INVENTION

Walking and running have been the main mobility mechanism for individuals, people and other mammals for ages. Some populations may benefit from tracking data related to those activities. Main populations that come to one's mind are athletes, elderly people, and people with various health condition states and disabilities.

A tracking solution by watch or a chest belt has some problems of convenience. Putting on and taking of such a belt my take time. Carrying a watch may be prohibitive or suboptimal in some group sports circumstances where it may put the participating parties in a risk of injury by the watch.

In addition, heart diseases and spread atrocities such as diabetes have been well documented to cause degradation of life quality to the extent of financial difficulties and burden on identified people who suffer from those diseases as well as. Monitoring health and addressing those result may proof beneficial to those who suffer from those diseases.

Nonetheless, some statistical studies of the recent years claim that movement of healthy adults may provide them with a better general health and longevity. Providing thereof means to stay active may help to achieve a better quality of living and may also help to spend less time and additional resources dealing with health treatments and their possible inconveniences.

### BRIEF SUMMARY OF THE INVENTION

Acknowledged herein is the necessity for an improved tracking system that is capable of addressing the needs of health and athletic populations as well as the general population. The increased prevalence of computation power as well as communication infrastructure and computational memory may provide opportunities for better health management and may also serve as a true motivator for athletes as well as general people to maintain a better health condition.

The present disclosure relates to systems, devices and methods for managing health and physical condition with a portable device. The disclosure relates to systems, devices and methods for managing health that is aggregated from the feet area with mobile computing or telecommunication devices.

The present disclosure further relates to systems, devices and methods for managing health and physical condition with a portable device on a moving individual. In particular, the present disclosure relates to systems, devices and methods for managing health that is aggregated from the feet area with mobile computing or telecommunication devices and then being analyzed by a computer system to asses the risk level to that individual.

Aspects of the present disclosure further relate to locating the device as such it will be in a position where it could communicate successfully and persistently over time with a remote communication port. An example of such communication is with a Global Positioning System station. Additional examples are a communication with local Wi-Fi port or Bluetooth (or both). Additional examples are a communication via radio signals to mobile phone stations. Those are none exclusive examples and any permutation of them or them with additional telecommunications protocols that may proof beneficial to the successful implementation of the invention described here thereof may apply.

Aspects of the present disclosure further relate to user interfaces and additional mechanism to share relevant data with an individual in such way that the said individual can get the information in a lucid manner either by graphical representation, or by sound, or by additional party who will manage the data for the said individual in case that it may find the said task and using the information overly laborious.

Aspects of the present disclosure further relate to harnessing the power of artificial intelligence including: machine learning, deep learning, and evolutional computing in order to recognize particular risk to the data that was aggregated by the system it's devices.

Embodiments of the invention relate to usage of ergonomic insole tracking device for tracking and monitoring of individual or groups of individuals participating in a common activity.

Embodiments of the invention relate to usage of ergonomic tracking device that is secured firmly into a designated space in a shoe other walking apparatus so that the original shape of the shoe or the said other walking apparatus is not deformed by the assemblage of the tracking device, so that the overall form remains substantially consistent. This assemblage is implemented with a designated mechanism for attaching securing and releasing the tracking device so that the tracking device stays secured inside the designated space in a firm manner as far as it is attached by the securing mechanism. The said designated place is located in the sole of the shoe/walking apparatus or in the heel side of the shoe/walking apparatus. The tracking device is utilized for tracking and monitoring of individual or groups of individuals participating in a common activity.

Embodiments of the invention relate to usage of patching or scotch mechanism for locating the tracking device in a location that is beneficiary for resilient monitoring of stationary and moving individuals

Embodiments of the invention relate to usage of the system as an alerting device to wake a stationary individual from an overly long stationary position and alert thereof of an activity that should be performed aiding to achieve better circulation, metabolism and general mobility.

Embodiments of the invention relate to usage of artificial intelligence, machine learning and predictive analysis, combined with a designated tracking device for tracking and monitoring of individual or groups of individuals participating in a common activity.

Embodiments of the invention relate to usage of triangulation effect for measuring a heart generated signals also known as Electrocardiographic signals [ECG] by sensors located at the feet with one or any of the aforementioned apparatuses.

An aspect of an embodiment of the invention relates to usage of a designated insole—hereinafter referred to as an insole apparatus (IA), individual having an interest or group of individuals that share a common interest that defines a common interest profile, upon their participation in an activity that required be tracked.

An aspect of an embodiment of the invention relates to usage of a adjusted into place (designated space) of a shoe, or other wearable tracking device, or other detachable device that is in proximity with the feet, tangent or connected to the feet—hereinafter referred to as a adjusted into place apparatus (AIPA), individual having an interest or group of individuals that share a common interest that defines a common interest profile, upon their participation in an activity that required be tracked.

An aspect of an embodiment of the invention, relates to providing “smart” sensors loaded connected device, hereinafter referred to as a smart feet system (SFS) that transmit data from the insole sensors to a nearby device by any communication protocol. SFS comprises an insole equipped with sensors to track health and athlete related parameters as well as the power apparatus and the transmitter apparatus.

SFS comprises an integrated insole shoe system and/or integrated shoe system which will allow the smart sensors to transmit general health and physical conditioned signal data over the air. An aspect of the system will allow the usage of it for tracking activity state of the user. In the form of example if one user may use the SFS and is performing a running activity, the device will transmit a regular signal into the cloud network and/or the receiving device software/application. The SFS system transmits relevant data that will be collected from the device such as speed and/or acceleration and/or heart rate and/or pedometer and/or impact and/or angles on the plain in which the feet contacts the ground (in order to track supination or overpronation) and etc. over the air for collecting and interpreting relevant data to the physical condition the user. The SFS system will also have a storage capacity to transfer the data after the activity phase into a mobile device or stationary device of any kind.

SFS comprises an integrated insole shoe system and/or integrated shoe system which will allow the smart sensors to transmit distress health and physical conditioned signal data over the air. An aspect of the system will allow the usage of it for tracking emergency state of the user. In the form of example if one user may use the insole and the weight is tracked by the sensor and yet no heart bit is being recognized, the device will transmit a distress signal into the cloud network and/or the receiving device software/application. The SFS system transmits relevant data over the air for collecting and interpreting relevant data to the physical condition the user.

SFS comprises an integrated insole shoe system or an integrated shoe system which will allow the smart sensors to be serialized and used for tracking group social activities that will allow the interpreting system to analyze common statistics of the common interaction the related to the activity. In such a case, a ball that is equipped with communication capabilities and a sensor is optional to be used with the SFS device to collect more data about the relative location of an individual and the ball.

### BRIEF DESCRIPTION OF FIGURES

Non-limiting examples of embodiments of the invention are described below with reference to the figure attached hereto and noted following this paragraph. Dimensions of components and features shown in the figure are chosen for convenience and clarity of presentation and are not necessarily shown to scale.

FIG. 1 schematically shows SFS providing tracking capability in accordance with the embodiments of the invention.

FIG. 2 schematically shows SFS providing tracking capability in accordance with the embodiments of the invention.

FIG. 3 schematically shows a side view of SFS, IA and AIPA providing tracking capability in accordance with the embodiments of the invention.

FIG. 4 schematically shows a side view of SFS and IA providing tracking capability in accordance with embodiments of the invention.

FIG. 5 schematically shows a side view of SFS and AIPA providing tracking capability in accordance with various embodiments of the invention.

FIG. 6 schematically shows SFS providing ECG measurement capability in accordance with various embodiments of the invention.

FIG. 7 schematically shows a flow chart of the AI capability in accordance to various embodiments of the invention.

FIG. 8 illustrates example of user interface that may be provided to indicate a first screen of an application in accordance with various implementations of the invention.

FIG. 9 illustrates example of user interface that may be provided to indicate a main screen of an application in accordance with various implementations of the invention.

FIG. 10 illustrates example of user interface that may be provided to indicate user's profile screen in accordance with various implementations of the invention.

### DETAILED DESCRIPTION OF THE INVENTION

portrayed herein are systems devices and methods for providing improved monitoring of an individual for either health related, athletic activity or everyday activity. Before going into depth and portraying an embodiment of the inventive concepts disclosed in detail, it is to be clear that the inventive concepts are not limited in their application to the details that are discussed in the descriptions or in their graphical representation. The disclosed inventive concepts include other embodiments or of being practiced or being practiced in varied ways. In addition, it is to be acknowledged that the terms and phrases employed herein are for the sake of description only and shall not be treated as limiting in any way.

In the further detailed description of embodiments of the described Subject matter, numerous specific details are set forth in order to provide a more thorough understanding of the inventive concepts. However, it will be apparent too one of ordinary skill in the art that the inventive concepts within the disclosure may be practiced without these specific details. In other instances, well-known features have not been described in detail to avoid unnecessarily complicating the instant disclosure.

Moreover, the usage of the term “or” anywhere in the given specification is inclusive as in the regular usage of the logical disjunctive expression unless noted otherwise. Thus, for any given two operators. If one of them is true the output is true. And if both operators are true the output is true as well. Further, use of the “a” or “an” are given to describe elements and components of the presented embodiments. This is done for convenience and to give a general sense of the inventive concepts. This description should be read to include one or at least one and the singular also includes the plural unless it is obvious that it is meant otherwise. Furthermore, as used herein, any reference to “one embodiment” or “an embodiment” means that a particular element, feature, structure, or characteristic described in connection with the embodiment is included in at least one embodiment. The appearances of the phrase “in one embodiment” in the specification are not necessarily all referring to the same embodiment. By the same token, the appearances of the phrase “in an embodiment” in the specification are not necessarily all referring to the same embodiment.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise various features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

In one embodiment of the invention a transitory tracking/sensing device is attached to the proximity of the feet and communicates signals sensed from that environment. The signals that are collected are of health and athletic importance. The signal is being kept in local memory and also is transmitted to a near by communication port that may be achieved by a router or a mobile device of any kind. The data transmitted is then stored in a remote server that interprets and compare the data to the data of the individual that it was related to originally and also to other individuals. Then, the system by means of machine and deep learning methods predicts the current and future risk level to the said individual and communicates it back to the user with recommendations for improvements or suggested training regime. The communication of the information relevant to the user can be directly sent to the user by a handy user interface by sound or graphical representation or be sent to additional party that will manage that information for the individual in some implementations. The System is further capable of communicating relevant data to healthcare centers if that is salubrious. The location of the sensing device is interchangeable. and can be attached and removed by the said individual, that may assist to provide a longer sustainability and usage time in the case of switching to a different walking device. The fact that biometric data is collected including by the feet is advantageous for the said individual because that set free the need to be obliged to attach itself to additional screen device and physical devices. Hence, the feet location is taking advantage of available space that is already being utilized regularly by many individuals who use walking or running devices regularly for the means of cushioning and feet protection. In this embodiment a patch mechanism inside a designated space of a shoe allows the placing and removal of the device from the shoe. The designated space is located as such the antenna of the device is constructing the outer edge of the hill so a communication to remote communication port such as GPS or mobile device such as tablet or smartphone or a router or mobile radio station's antenna or other communication port is facilitated by this location. This said embodiment mobile or smart watch or sound centric application include an option of the individual to share its results with other individual who use the system and that will aid to provide for some individuals an additional motivator to keep progress, maintain and improve overall condition.

In an embodiment of the invention, the user receive data via several methods that are transmitting methods including any form of on air transmitting into any device that will be able to receive such transmission. Including personal computer, smart watch, smart phone or any other designated device.

In an embodiment of the invention, a group of users may use the transmitter to track their relative locations and interactions: speed, location, and in the case of ball sports, ball possession into a collection terminal that will be able to analyze the group interaction. That group traction system will be transmitting into smart phone, smart watch or any other device.

Computer system and its components may be proprietary and may comprise servers and/or programming code located in a same location or may be a distributed system having, optionally non-proprietary, program code and/or servers located in different locations that are accessed through the internet (“cloud based”) or via IR, NFC WLAN, Bluetooth or any other communication protocol. For instance, people and profiles data base may comprise information from a social network data base maintained on one server while activity venues data base may be maintained by, optionally a local event company, in a data base on another server.

Optionally, people may manage and/or maintain information relating to their interest profiles on their mobile devices, optionally by installing an application that updates their interests and activities and/or that may retrieve such information via the INTERNET CLOUD from a web-based data base. Upon gathering at an activity venue their mobile devices provide their interest profiles in addition to their geolocation.

Whereas in the previous paragraph interest groups are described as gathered at different activity venues, there may of course be a plurality of interest groups present at a same activity venue.

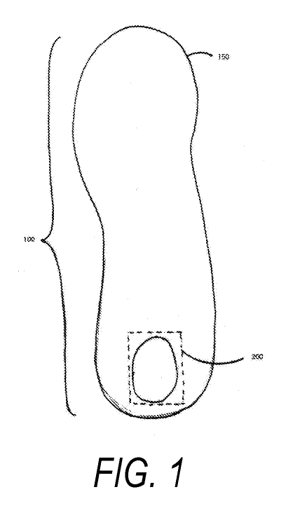

In the following detailed description, a SFS 100 in accordance with an embodiment of the invention for tracking physical conditions as well as movement (distance, steps, steps distance and acceleration, force of landing, impact, tilt angle, etc.) and heart condition with reference to FIG. 1.

FIG. 1, 100 schematically exemplify an insole device 150 and within the insole device an area 200 illustrated by a dashed line comprising the tracking and transmitting device, hereinafter also referred as TD. Optionally a transmitting beacon, hereinafter also referred to as a “transmitting beacon device,” is attached to the Insole. By way of example, TD 200 is attached to the Insole 150. The location in which the

TD is located at any area within the Insole and that is to clarify that FIG. 1 portray only one valid location for TD amongst any. It may also take in size the whole insole or some of it. The TD stores and tracks physical information about the user of the device and the ways which in they interact with it. It may be able to account for various parameters amongst which heart rate, speed, distance, acceleration, physical impact, attack, contact duration with ground, geolocation, and more.

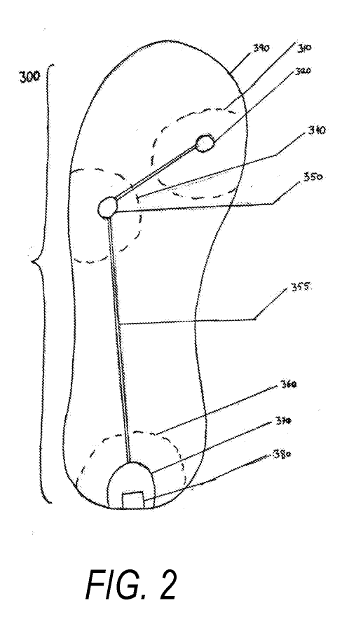

FIG. 2, 300 schematically shows an insole device 390 with areas 310,340, 360 respectively illustrated by a dashed line comprising the tracking devices 320,350, 370, hereinafter also referred as TDs. Optionally a transmitting beacon, hereinafter also referred to as a “transmitting beacon device,” is integrated into the Insole. By way of example, TD 370 is attached to the Insole 390. The location in which the TD is located at any area within the Insole and that is to clarify that FIG. 1 portray only one valid location for TD amongst any. It may also take in size the whole insole or some of it. The tracking and transmitting devices 320,350,370 are exemplified in FIG. 2, whereby the communication interface 355 is exemplified as well.

The integrated circuit 380 schematically drawn in FIG. 2 is located at the edge of the insole 390 in order to facilitate the signal transmission into the target communication device.

FIG. 2 schematically show an installation in which the sensors location is in accordance with the nerve supply of the sole: the tibia 310, the lateral plantar 340, and the medial plantar 360. This distribution allows for an accurate measurement of the feet's health and athletic medical data.

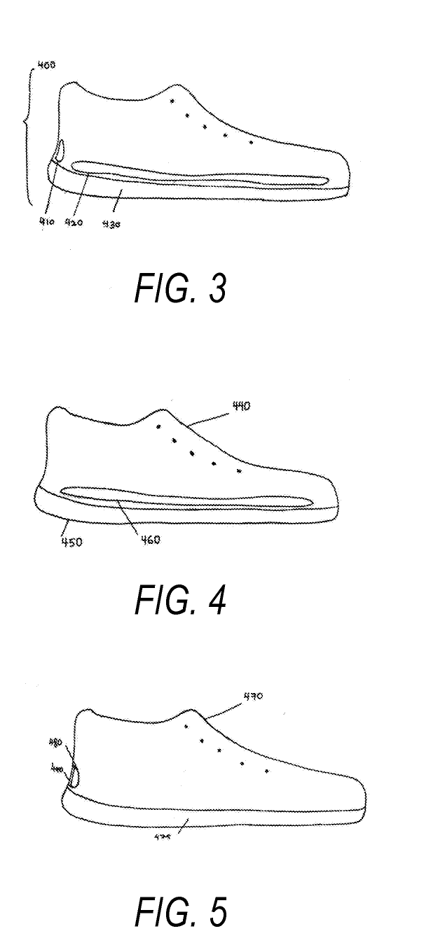

FIG. 3 schematically shows a shoe 400 with distinctive parts: an area for TD in the back of the heel 410, an area for TD in the insole of the shoe 420, and a sole of the shoe 430. The usage of TD in the back of the heel 410 is potentially by enablement of placing in a transitory device which can be afterwards displaced and used in an additional shoe. The transitory device potentially adjusted into place from the either side of the shoe: internal or external. The usage of TD in the back of the heel 410 is potentially by enablement of placing in a non-transitory device which integrated in the manufacturing process and cannot be afterwards displaced and used in an additional shoe. The implementation of TD in the back of the heel 410 is located at the edge of the shoe 400 in order to facilitate the signal transmission into the target communication device. This configuration include a TD located in the back of the heel area 410, or a TD located in the insole of the Shoe 420 or both (inclusive or).

FIG. 4 schematically presents an embodiment of the invention with a walking device 440, with distinctive parts: an area for TD in the insole of the shoe 460, and a sole of the shoe 450.

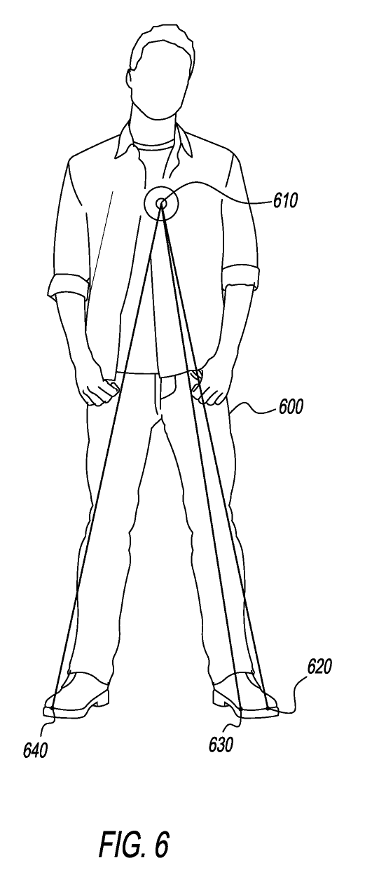

FIG. 5 schematically presents an embodiment of the invention with a walking device 470, with distinctive parts: an area for TD in the back of the heel 480, a surface that is in the outer edge of the walking device's back of the hill area 490, and a sole of the shoe 475.

FIG. 6 schematically shows an apparatus for measuring electrocardiographic signal via the feet. A human FIG. 600 is schematically shown in a stance that facilitate the exemplification of the apparatus. The bodily heart 610 is transmitting signal that is sensed by the TDs 620,630,640. For example, a heart 610 with TD 620 and additional TD 640 create a triangle which allow for measuring of ECG. Additional example is in the formation of another triangle that forms in only in one foot with heart 610 and two TDs 620,630. Additional example is in the formation of another triangle that forms in only in one foot with heart 610 and one TD 620, in which that TD 620 in that formation contains two remote sensors that enable a triangle. The form of triangle is not limited to a particular location in the feet. And for example, could be achieved in the heal side of the feet.

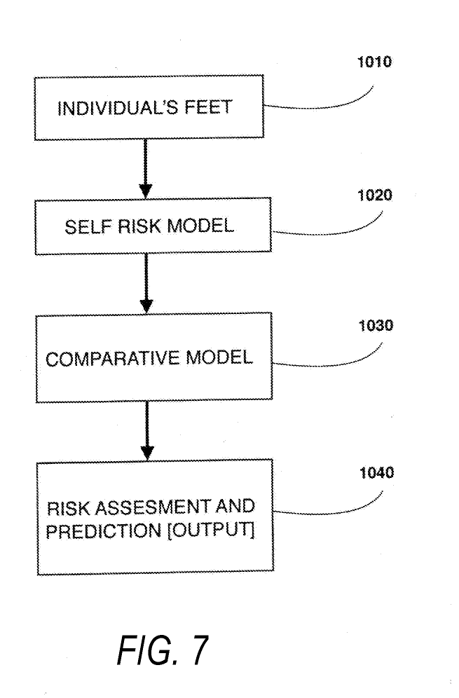

FIG. 7 schematically shows an embodiment of a flow chart which describe the general machine learning process that will be understood and constructed by any person who is skilled in the art. In the given figure it is shown that the data collected from an individual 1010 is then being assessed to be reviewed against the own individual's data in a module that is titled “Self Risk Model” 1020, in this action assessment is made for extreme changes and against known risk levels of information that is available. For example, a dangerous level of feet pronation, or heart rate pattern. Additionally, the said individual is compared with a set of relevant individuals from a data pool 1030 to review if they have intersecting of risk factors so that other individuals' risks may apply to the said individual. A final risk assessment 1040 is than performed in order to validate the recommendation and exclude false warnings.

In another embodiment of the flow chart of FIG. 7 the Risk assessment is only being done at the final and the self risk model 1020 and comparative model serve as means for data collection that will be analyzed together at the “Risk Assessment and Prediction [Output]” stage 1040. This assessment is then continuously checked against the current level of prediction so error margin is continuously diminished as more data is available for the prediction system. So that the alerts given by the system prediction 1040 become more accurate.

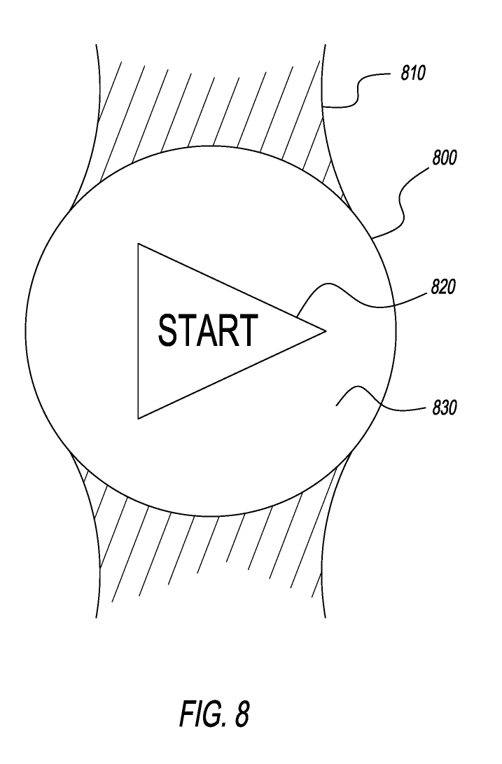

FIG. 8 illustrate example of user interface that may be provided to indicate a first screen of an application according to various implementations of the invention.

In FIG. 8. a smart watch 800, is attached to a strip 810, and the screen of the smart watch 830 is depicting a view with a single element 820 in order to facilitate the usage of the application and to avoid being overwhelmed by too many elements in the view. Additionally, a background process may be executing (running) in the background in order to activate the application when a relevant activity is performed by an individual.

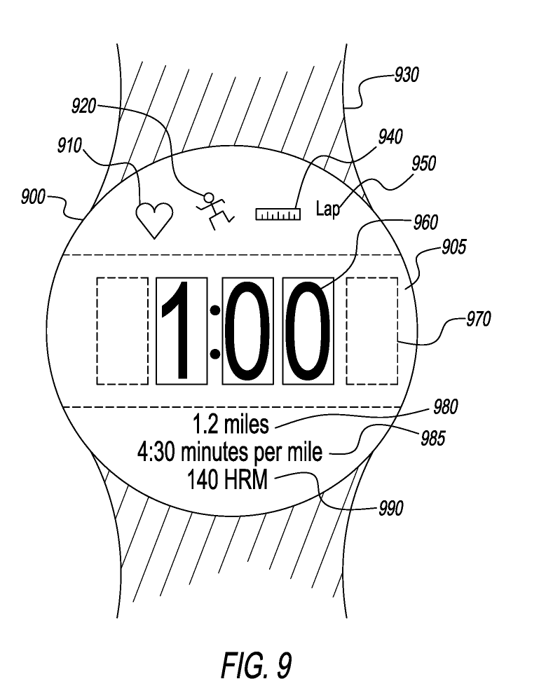

FIG. 9 illustrate example of user interface that may be provided to indicate a main screen of an application according to various implementations of the invention. In FIG. 9. a smart watch 900, is attached to a strip 930, and the screen of the smart watch 905 is depicting main view of an embodiment of an application during an activity. The lined prescribed box 960, delineate a second digital representation and the ones to it's right delineates ten seconds and minute respectively. The outer dashed line prescribed box 970, delineate optional view of tens of second and the left side outer dashed line box represents optional view of tens of minutes that is being shown after ten minutes of activity have been achieved. The heart icon 910, is part of the top menu and represents heart rate of the individual using the application it may be animated to grow and shrink simultaneously with the heart bit of the said individual. A push on the heart icon may open additional view of heart rate related data. The running figure icon 920 is also potentially animated as such the running figure inclination is matched to the speed of the individual using the application. A push on the running figure icon may open additional view of running related information such as speed stride length and so forth. The ruler icon 940, represents distance and a click on it may open an additional view of distance related information such as distance, altitude and terrain information. A “Lap” icon 950 may represent number of laps or be replaced or added by a map icon that may be also added to all the other icons in the top menu but is not drawn in FIG. 9. for simplifying reasons. In the bottom section of the view there are optional text graphic that help to orient the user on the state of the activity sessions. the text view 980 represents distance. While the text view 985 depict speed rate in minutes per unit where unit is a distance unit such as mile or kilometer and the bottom text view 990 limns the heart rate of an individual that is using the app. Optionally, the user can reorder those text view and for example replace the bottom view with the top view. The user can and also switch between bottom and upper menus in the settings or preferences of the watch application.

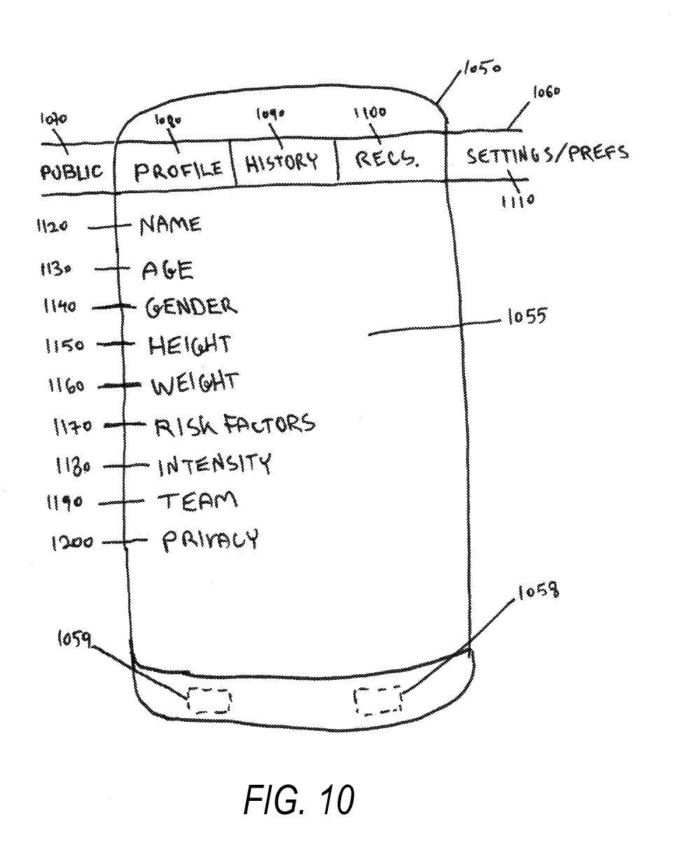

FIG. 10 illustrate example of user interface that may be provided to indicate user's profile screen according to various implementations of the invention. In FIG. 10. an application of a mobile device 1050, such an android mobile phone or an iPhone or a tablet computing device. A top menu 1050, optionally scrollable is further depicted in the Figure. The profile view 1080, is portrayed in this illustration on the screen of the device 1055, the fields of the profile view which are given respectively are: name, age, gender, height, weight, risk factors, intensity, team, and privacy (1120,1130, 1140, 1150, 1160, 1170, 1180, 1190, 1200). Risk factors 1170 include risk factors identified by user and also risk factor that were revealed by the system and where unknown to the user previously. Additional viewed in the application are: Public 1070, which will allow a user to share information publicly and compare results with friends and other people of interest that allow from their side to share and compare results, that may serve as motivator for some users and aid to improve their condition. History view 1090, is giving relevant data at previous sessions such as distance covered and feet pronation angles impact and map of the covered course by the session. Recs. view 1100, is an abbreviation for recommendations and in it the user is giving recommendations and suggestions by the system to improve its form. Recommendations may also appear in other screens. Settings/prefs view 1060, is where user can set various options regarding to the behavior of the application such as automatic operation in the background of the device, enablement and disablement of sound, choosing a training program that can focus on: strength, stamina, free, intensity levels, agility, distance covered per day, and simple monitoring activity for healthcare review and assistance. That view 1060, may also include an activity alert which when activated remind by the way of sound vibration notification or other alert mechanism that the user has been stationary for elongated period (that may be set in this view also) and it's for the user to move and perform a physical activity. Right speaker 1058 and left speaker 1059, are further depict in order to provide sound output that could be used by a user using the application. Potentially by giving key signals about intensity of training and reaching a pre-set target.

What is claimed is:

1. An apparatus for tracking health and physical condition via foot, comprising:
a. a device that activates at least one sensor to track health and movement of the feet and other body signals;
b. a package to contain the said electric circuit;
c. a power unit to supply energy to the device that is either operated by cell energy exclusively, or by the energy collected by motion of an individual exclusively, or by solar energy exclusively, or by a combination of the given power mechanisms;
d. a designated vacant space in a walking device to place the said package, or
e. an insole component that contains the said package and the said package, or
f. a removable unit for holding the said device in assembled to the said package when it is elastically assembled around an individual body.

2. A mechanism to store and remove the said apparatus from 1, comprising a designated housing space inside a walking device that can contain the said package of said transitory apparatus.

3. A remote unit to store the data collected by the said apparatus from 1, comprising:
a. a transmitting/receiving component to get and send data to other means of communication,
b. a memory unit to store the data,
c. a processing unit to perform calculations as well as simple operations to assert valid data is maintained,
d. a mechanism to split the data within the unit by groups,
Whereby the remote unit has the ability to share the obtained data for health, sports, and social activities and medical tracking as well as athletic tracking and review.

4. The said apparatus from 1, with a sensing device that measures:
(a) the impact of foot or the walking device contact with the ground,
(b) the tilt angel of the foot when the foot hits the ground.

5. The said apparatus from claim 1 wherein the said transmitting/receiving component is attached to a signaling mean that is closer to outer surface by less than 11 millimeters so that a communication with a remote signal receiver is eased by the particular location of the signaling mean.

6. A set of the said apparatus from 1, when an additional apparatus from 1 is used in conjunction with the first said apparatus on a different foot of an individual. Whereby, the data that is collected from the set is potentially used for comparing method in order to track discrepancies in walking and running, and also to track athletic performance.

7. The said apparatus from 1, further including a mechanism for alerting. Whereby, the device transmits a signal to nearby communication ports in order to facilitate a rescue of a distressed individual.

8. A method for measuring health signals via the feet, comprising:
a. sensing the feet environment;
b. preparing the data locally for transmission;
c. transmitting digital signals into a communication device;
d. storing the signals for further usage;
e. interpolating the data received,
Whereby, advantage of given method is the location of the measurement in an area that is already covered by material. Hence does liberate an individual from a burden of having to handle a specialized device like a watch or a chest belt for tracking health and fitness levels.

9. The method of claim 8, further including:
(a) interpreting the data according to standard performance and self comparison,
(b) providing related tips and suggestions to group and individuals regarding the current state and interpretation,
(c) sharing the information forming a group of individuals.

10. The method of claim 8, further including:
(a) detecting a distress activity in the proximity of the device,
(b) sending a respective distress signal to due recipient.

11. The method of claim 8, wherein health signals measured are ECG signals.

12. The method of claim 8, wherein health signals measured are ECG signals, and achieving ECG signal triangulation is achieved with one foot or with two feet.

13. The method of claim 8, wherein health signals measured by means of remote sensing,
Whereby, the measuring sensor is remoted from the body surface of an individual so a signal is moving through a material.

14. The said method from 10, where signals are sent to a remote unit where:
a. the said signals are aggregated and being stored as data,
b. a calculation on the said data for addressing level of risk factors for the signal's source is being made,
c. the calculation is being transmitted back to a display unit.

15. The said method from 9, where signals are sent to a remote unit where:
a. the said signals are aggregated and being stored as data,
b. a calculation on the said data for predictive data of the signal's source is being made,
c. a calculation on the said predictive data for addressing level of risk factors for the signal's source is being made,
d. the said calculation's result is being transmitted back to an end user.

16. A system to store and share physical health data that is collected from the feet area, comprising:
a. a sensing device for collecting data located at the feet area up to approximately ten centimeters from the bottom edge of the walking device's sole,
b. a transmitting unit which pass the data collected from the said sensing device,
c. a memory unit for storing the said physical health data,
d. a central unit that stores the data in the said memory unit and retrieves it for further operations.

17. A calculation unit of the system from 16, where the said calculating unit performs additional calculations comprising a timely calculation of risk to individuals based on the individual's data combined with standard health procedures and schemes.

18. A calculation unit of the system from 16, where the said calculating unit performs additional calculations comprising a predictive calculation of individual's data based on the individual's data combined with Machine learning predictive methods.

19. A calculation unit of the system from 16, where the said calculating unit performs additional calculations comprising a predictive calculation of risk to individuals based on the individual's data combined with standard health procedures and schemes.

20. A memory unit of the system from 16, where the data contained by the said memory unit is separated for special groups of individuals, whereby the data is used to enhance the group performance or knowledge about their physical and health condition.
